(19) United States
(12) Patent Application Publication
(10) Pub. No.: US 2026/0183215 A1
(43) Pub. Date: Jul. 2, 2026

(54) PRESERVATIVE-FREE CLEANSING AND CARING GRANULE WITH LOW WATER ACTIVITY AND PREPARATION METHOD THEREOF

(71) Applicant: Guangzhou Beaver Cosmetics Co., Ltd., Guangzhou (CN)

(72) Inventor: Hongpeng Zhang, Guangzhou (CN)

(21) Appl. No.: 19/257,420

(22) Filed: Jul. 1, 2025

(30) Foreign Application Priority Data
Dec. 30, 2024 (CN) 202411965575.7

(51) Int. Cl. A61K 8/73; A61K 8/02; A61K 8/34; A61K 8/36; A61K 8/365; A61K 8/41; A61K 8/44; A61K 8/46; A61K 8/60; A61K 8/9728; A61K 8/9789; A61K 8/9794; A61K 8/99

(52) U.S. Cl. CPC A61K 8/731; A61K 8/0225; A61K 8/345; A61K 8/361; A61K 8/365; A61K 8/416; A61K 8/44; A61K 8/463; A61K 8/466; A61K 8/60; A61K 8/732; A61K 8/735; A61K 8/736; A61K 8/9728; A61K 8/9789; A61K 8/9794; A61K 8/99; A61K 2800/30; A61K 2800/48; A61K 2800/596; A61K 2800/624; A61K 2800/654; A61K 2800/805; A61K 2800/85

(57) ABSTRACT

Provided are a preservative-free cleansing and caring granule with low water activity and a preparation method thereof. The preparation method includes the following steps: S1, mixing other components than a thickener evenly to give a mixture, then subjecting the mixture to drying and grinding to give powder; S2, mixing the thickener with water to give a thickener aqueous solution, then adding the powder obtained in step S1 to a granulator, and spraying the thickener aqueous solution obtained in step S2 into the granulator to prepare a granule as the cleansing and caring granule, during which an air temperature in the granulator is controlled by a blower; where a mass concentration of the thickener aqueous solution is 0.3%-0.7%, and the air temperature in the granulator is controlled at 80° C.-90° C.

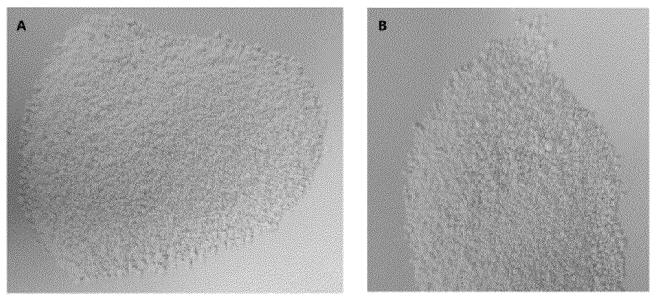

### CROSS-REFERENCE TO RELATED APPLICATION

This disclosure claims the benefit of Chinese patent application No. 202411965575.7, filed on Dec. 30, 2024, which is incorporated herein by reference in its entirety.

### TECHNICAL FIELD

This disclosure belongs to the technical field of daily chemical products, and in particular relates to a preservative-free cleansing and caring granule with low water activity and a preparation method thereof.

### BACKGROUND

Currently, traditional liquid cleansing and caring products such as shampoo and shower gel contain certain chemicals, especially preservatives, which may irritate a user's skin and cause allergic reactions, thereby affecting the user's comfort level and health. In addition, the production and packaging processes of liquid cleansing and caring products usually require a large amount of water, which not only increases production costs, but also imposes a burden on the environment. Especially in today when global water resources are increasingly strained, how to reduce the water consumption in the production process has become an urgent challenge for the cleansing and caring industry.

Accordingly, developing a specific method for preparing a preservative-free solid cleansing and caring granule has become an important direction of market demand. The solid cleansing and caring granule can not only reduce the requirement for chemical ingredients, avoiding allergies and irritation problems, but also reduce water consumption in production and transportation by simplifying the formulation and replacing with convenient packaging. In addition, the granular cleansing and caring product can be dissolved only by adding a little water when used, reducing the dependence of traditional liquid cleansing and caring products on water resources. It is also easier to perform accurate dose control, avoiding waste. Meanwhile, the unique granular shape also helps to extend the shelf life of the product, reduce the requirement for preservatives, and further improve the safety and environmental protection property of the product.

### SUMMARY

An objective of this disclosure is to overcome the disadvantages of the prior art and provide a preservative-free cleansing and caring granule with low water activity and a preparation method thereof.

To achieve the above objective, the following technical solutions are adopted.

In a first aspect, provided herein is a preparation method of a preservative-free cleansing and caring granule with low water activity, comprising the following steps:
S1. weighing the following components in parts by weight: 22-61 parts of a surfactant, 10-15 parts of a filler, 1-5 parts of an anti-caking agent, 2.1-21 parts of a moisturizer, 0.1-1 part of a pH regulator and 0.5-1.5 parts of a fragrance, mixing the weighed components evenly to give a mixture, subjecting the mixture to drying and grinding to give powder;
S2. weighing a thickener, and mixing the thickener with water to give a thickener aqueous solution;
S3. adding the powder obtained in step S1 to a granulator, then spraying the thickener aqueous solution obtained in step S2 into the granulator to prepare a granule as the cleansing and caring granule, during which an air temperature in the granulator is controlled at 80° C.-90° C.;
wherein a mass concentration of the thickener aqueous solution is 0.3%-0.7%.

The disclosure adopts the above specified method to prepare a solid cleansing and caring granule by using the selected components. By spraying the thickener aqueous solution onto the surface of the powder prepared from the components except for the thickener to form a solid granule, the adhesiveness and uniformity among the various components can be effectively improved, and a film can be formed on the surface of the granule to wrap the granule to a certain extent, so as to prevent water from penetrating too quickly and reduce water evaporation. Thus, the water activity of the cleansing and caring granule can be effectively reduced.

Through experimental research, it has been found that in the preparation method, the concentration of the thickener aqueous solution and the air temperature during granulation have an important impact on the formability and water activity of the final cleansing and caring granule. When the concentration of the thickener aqueous solution is too low, the binding force among the components will be weak, resulting in uncompact granules, thus non-uniform or uncompact and fragile granules will be produced; while when the concentration of the thickener aqueous solution is too high, the viscosity of the thickener aqueous solution is high, which will lead to the reduction of the fluidity of the granules, affecting the uniformity and dispersion of the granules. When the air temperature during granulation is too low, it is difficult to dry the granules completely, resulting in low hardness, high moisture absorption, and high water activity; when the air temperature during granulation is too high, the surface of the granules will be hardened and a large amount of water is kept inside the granules, thus affecting the uniformity and formability of the granules. In addition, a high temperature may also affect the performance of the thickener, resulting in degradation or altered properties of the thickener, thus affecting the structure and stability of the granules.

Therefore, the disclosure adopts the above specified method to prepare the cleansing and caring granule. Under a synergistic effect of the optimized thickener concentration and granulation temperature, the disintegration or deformation of the granules is effectively avoided, thereby ensuring the size uniformity of the granules. Simultaneously, the water activity of the granules is significantly reduced, so as to ensure the quality and stability of the preservative-free cleansing and caring granule.

Preferably, a mass ratio of the thickener aqueous solution to the powder in step S3 is (0.2-0.4):1.

More preferably, the mass ratio of the thickener aqueous solution to the powder is 0.3:1.

In the preparation process of the cleansing and caring granule, the mass ratio of the thickener aqueous solution to the powder is also an important factor to control the quality and performance of granules. When the two are combined at the above optimized mass ratio for granule preparation, the formation of ideal granule structure from the components and uniform granule size distribution can be achieved, which effectively improves the mechanical strength of granules, reduces the risk of breakage and fragmentation of granules during storage or transportation. In addition, the solubility of the granules and hydration during use can be ensured to a certain extent, thus providing good sensory experience for consumers.

Preferably, in step S3, the air temperature in the granulator is controlled by a blower with an operating frequency of 15-50 Hz; and/or, an outlet air temperature of the granulator is controlled at 50° C. or lower, and a humidity is controlled at 20-40%.

Through experimental research, it has been found that the operating frequency of the blower and humidity of the granulator during granulation have an important impact on the shape, structure and stability of the granules. If the parameters exceed the above optimized ranges, the uniformity and integrity of the granules will be adversely affected, resulting in that the granules cohere to one another or too small granules agglomerate. Especially when the humidity is too low, it will cause the granules to break easily or fail to form the expected shape, while too high humidity may cause the granules to easily agglomerate, thus affecting the shape and fluidity of the granules. Therefore, when the cleansing and caring granule is prepared, the precise control of each parameter can effectively ensure the quality, stability and final usage effect of the granules.

More preferably, the air temperature inside the granulator is controlled at 85° C., the outlet air temperature of the granulator is controlled at 30° C., and the humidity is controlled at 30%.

Preferably, the mass concentration of the thickener in the thickener aqueous solution is 0.5%.

Preferably, after the granules are produced, target granules within a 10-100 mesh range are sieved to give the cleansing and caring granules.

Preferably, the preservative-free cleansing and caring granule comprises the following components in parts by weight: 45 parts of the surfactant, 12 parts of the filler, 3 parts of the anti-caking agent, 15 parts of the moisturizer, 0.5 parts of the pH regulator and 1 part of the fragrance.

Through experimental research, it has been found that under the same preparation process conditions, when the components are combined at the optimized ratios for the preparation of the cleansing and caring granule, the components can cooperate with one another to reduce the water activity of the cleansing and caring granule to the greatest extent and improve the usage performance of the granule, and the long-term stability of the granule can be effectively guaranteed without adding preservatives.

Preferably, the thickener comprises hydroxypropyl methyl cellulose, xanthan gum and chitosan, and a mass ratio of the hydroxypropyl methyl cellulose/xanthan gum/chitosan is (0.1-1):(0.1-0.5):(0.1-0.3).

More preferably, the mass ratio of the hydroxypropyl methyl cellulose/xanthan gum/chitosan is 0.5:0.3:0.2.

For the preparation of the cleansing and caring granule, the inventors also researched the selection of the thickener. Through experimental research, it has been found that when hydroxypropyl methyl cellulose, xanthan gum and chitosan are combined in the above optimized ratio range as a thickener for the preparation of the cleansing and caring granule, the three may interact with one another, which can effectively avoid the problem of too slow dissolution rate or non-uniform granules caused by a thickener composed of one single component. By adjusting the viscosity of the thickener aqueous solution, the structure and performance of the granule can be further improved, so as to significantly improve the stability of the granules. In addition, the antibacterial property of chitosan can complement the properties of the other two thickener components, which helps to increase the antibacterial property of the cleansing and caring granule without adding preservatives and further strengthen the long-term stability of the granule.

Preferably, the surfactant comprises cetyltrimethyl ammonium chloride, cocamidopropyl betaine, sodium lauryl sulfate and sodium cocamyl hydroxyethylsulfonate; and a mass ratio of cetyltrimethyl ammonium chloride/cocamidopropyl betaine/sodium lauryl sulfate/sodium cocamyl hydroxyethylsulfonate is (5-20):(5-15):(10-20):(2-6).

More preferably, the mass ratio of cetyltrimethyl ammonium chloride/cocamidopropyl betaine/sodium lauryl sulfate/sodium cocamyl hydroxyethylsulfonate is 15:10:15:5.

In this disclosure, when the above four surfactant components are combined in the above specified ratio range, the four complement with one another, so that the cleansing and caring granule can not only efficiently clean the hair and scalp, but also avoid the irritation and dryness that may be brought by a surfactant composed by one single component, thereby achieving a mild and deep cleansing effect. In addition, the combination of the four surfactant components can collaboratively improve the richness, stability and fineness of the foam, and a good rinsing effect of the cleansing and caring granule after use can be maintained, avoiding incomplete rinsing, so as to provide consumers with better usage experience.

Preferably, the filler comprises at least one of a modified corn starch, microcrystalline cellulose and polyvinyl alcohol; and/or, the anti-caking agent comprises magnesium stearate or silicon dioxide; and/or, the moisturizer comprises at least one of sodium hyaluronate, trehalose and sorbitol; and/or, the pH regulator comprises citric acid; and/or, the fragrance comprises an essence.

In a second aspect, provided herein is a use of the preparation method in preparing a cleansing and caring product, wherein the cleansing and caring product is selected from a shampoo product, a bath product and a facial cleanser product.

The preparation method of the disclosure can be widely used in the preparation of a personal care solid granule product, such as a shampoo product, a facial cleanser product or a bath product. The method can reduce the water activity of the cleansing and caring granule to the greatest extent and improve the usage performance of the granule, effectively ensure the long-term stability of the granule without preservatives added, and effectively avoid the irritation or allergic reaction brought by preservatives to the skin. The obtained personal care solid granule product is suitable for all kinds of skin, especially sensitive skin.

In a third aspect, provided herein is a preservative-free cleansing and caring granule with low water activity, wherein the cleansing and caring granule comprises the cleansing and caring granule obtained by the above preparation method, a hair regulator and a softener.

Preferably, the softener comprises at least one of acanthus kernel oil, Jojoba seed oil and coconut oil; and the hair regulator comprises Lactobacillus/rye flour ferment filtrate.

Compared with the prior art, this disclosure has the following beneficial effects.
(1) In this disclosure, a solid cleansing and caring granule is innovatively prepared by adopting the specified preparation method. By precisely controlling various important parameters in the method, the formation of an ideal granule structure and uniform granule size distribution can be ensured, which effectively improves the mechanical strength of the granule, reduces the risk of breakage and fragmentation of the granule during storage or transportation. The preparation method of the disclosure can significantly reduce the water activity of the granule, thus ensuring the quality, stability and overall usage performance of the obtained preservative-free cleansing and caring granule. In addition, the obtained preservative-free cleansing and caring granule is easy to rinse without residues, significantly reducing water consumption and the consumption of water resources. Furthermore, the obtained preservative-free cleansing and caring granule is easy to transport or carry due to a greatly reduced volume.
(2) In the disclosure, the specified surfactant and the thickener are combined with other components to prepare the cleansing and caring granule. These components cooperate with one another to effectively achieve a mild and deep cleansing effect of the cleansing and caring granule, and collaboratively improve the richness, stability and fineness of the foam, while improving the structure and performance of the granule to further improve the long-term stability of the granule. Meanwhile, a good rinsing effect of the cleansing and caring granule after use is maintained, which effectively improves the user's usage experience.

### BRIEF DESCRIPTION OF THE DRAWINGS

The sole FIGURE shows the appearance images of the cleansing and caring granules obtained in Example 1 (A) and in Comparative Example 1 (B).

### DETAILED DESCRIPTION

To better explain the objectives, technical solutions and advantages of the disclosure, specific examples are provided to further explain the disclosure. It should be understood by those skilled in the art that the specific examples are provided only to explain the disclosure, and are not intended to limit it.

Raw materials involved in the following examples and comparative examples are commercially available materials, unless otherwise specified. The experimental operations not specifically stated herein are routine operations in this field, or can be understood or known by those skilled in the art based on prior art or common knowledge.

### Example 1

A preparation method of a preservative-free cleansing and caring granule with low water activity was provided in Example 1, comprising the following steps.
S1. the following components were weighed in parts by weight: 15 parts of cetyl trimethyl ammonium chloride, 10 parts of cocamidopropyl betaine, 15 parts of sodium lauryl sulfate, 5 parts of sodium cocamoyl hydroxyethionate, 12 parts of modified corn starch, 3 parts of magnesium stearate, 1 part of sodium hyaluronate, 9 parts of trehalose, 5 parts of sorbitol, 0.001 parts of Lactobacillus/rye flour ferment filtrate, 0.001 parts of acanthus kernel oil, 0.001 parts of Jojoba seed oil, 0.001 parts of coconut oil, 0.5 parts of citric acid and 1 part of daily essence; the weighed components were mixed evenly to give a mixture, and the mixture was dried and ground into powder;
S2. hydroxypropyl methyl cellulose, xanthan gum and chitosan were weighed as a thickener, and the thickener was mixed with water to give a thickener aqueous solution with a mass concentration of 0.5%, where a mass ratio of hydroxypropyl methyl cellulose/xanthan gum/chitosan was 0.5:0.3:0.2;
S3. the powder obtained in step S1 was added to a granulator, and then the thickener aqueous solution obtained in step S2 was evenly sprayed into the granulator by using a peristaltic pump, an air temperature of the granulator was controlled at 85° C. by a blower with an operating frequency of 15 Hz, so that the powder was turned up and down in the granulator to fully contact with the sprayed thickener aqueous solution to produce granules, where the outlet air temperature of the granulator was controlled at 30° C. and the humidity was controlled at 30%;
S4. target granules within a 10-100 mesh range were sieved as the cleansing and caring granule for filling to give the finished product;
wherein in step S3, a mass ratio of the thickener aqueous solution to the powder was 0.3:1.

### Example 2

A preparation method of a preservative-free cleansing and caring granule with low water activity was provided in Example 2, comprising the following steps.
S1. the following components were weighed in parts by weight: 15 parts of cetyl trimethyl ammonium chloride, 10 parts of cocamidopropyl betaine, 15 parts of sodium lauryl sulfate, 5 parts of sodium cocamoyl hydroxyethionate, 12 parts of modified corn starch, 3 parts of magnesium stearate, 1 part of sodium hyaluronate, 9 parts of trehalose, 5 parts of sorbitol, 0.001 parts of Lactobacillus/rye flour ferment filtrate, 0.001 parts of acanthus kernel oil, 0.001 parts of jojoba seed oil, 0.001 parts of coconut oil, 0.5 parts of citric acid and 1 part of daily essence; the weighed components were mixed evenly to give a mixture, and the mixture was dried and ground into powder;
S2. hydroxypropyl methyl cellulose, xanthan gum and chitosan were weighed as a thickener, and the thickener was mixed with water to give a thickener aqueous solution with a mass concentration of 0.7%, where a mass ratio of hydroxypropyl methyl cellulose/xanthan gum/chitosan was 1:0.1:0.1;
S3. the powder obtained in step S1 was added to a granulator, and then the thickener aqueous solution obtained in step S2 was evenly sprayed into the granulator by using a peristaltic pump, an air temperature of the granulator was controlled at 80° C. by a blower with an operating frequency of 50 Hz, so that the powder was turned up and down in the granulator to fully contact with the sprayed thickener aqueous solution to produce granules, where the outlet air temperature of the granulator was controlled at 50° C. and the humidity was controlled at 20%;
S4. target granules within a 10-100 mesh range were sieved as the cleansing and caring granules for filling to give the finished product;
wherein in step S3, a mass ratio of the thickener aqueous solution to the powder was 0.2:1.

### Example 3

A preparation method of a preservative-free cleansing and caring granule with low water activity was provided in Example 3, comprising the following steps.
S1. the following components were weighed in parts by weight: 15 parts of cetyl trimethyl ammonium chloride, 10 parts of cocamidopropyl betaine, 15 parts of sodium lauryl sulfate, 5 parts of sodium cocamoyl hydroxyethionate, 12 parts of modified corn starch, 3 parts of magnesium stearate, 1 part of sodium hyaluronate, 9 parts of trehalose, 5 parts of sorbitol, 0.001 parts of Lactobacillus/rye flour ferment filtrate, 0.001 parts of acanthus kernel oil, 0.001 parts of jojoba seed oil, 0.001 parts of coconut oil, 0.5 parts of citric acid and 1 part of daily essence; the weighed components were mixed evenly to give a mixture, and the mixture was dried and ground into powder;
S2. hydroxypropyl methyl cellulose, xanthan gum and chitosan were weighed as a thickener, and the thickener was mixed with water to give a thickener aqueous solution with a mass concentration of 0.3%, where a mass ratio of hydroxypropyl methyl cellulose/xanthan gum/chitosan was 0.1:0.5:0.3;
S3. the powder obtained in step S1 was added to a granulator, and then the thickener aqueous solution obtained in step S2 was evenly sprayed into the granulator by using a peristaltic pump, an air temperature of the granulator was controlled at 90° C. by a blower with an operating frequency of 50 Hz, so that the powder was turned up and down in the granulator to fully contact with the sprayed thickener aqueous solution to produce granules, where the outlet air temperature of the granulator was controlled at 50° C. and the humidity was controlled at 40%;
S4. target granules within a 10-100 mesh range were sieved as the cleansing and caring granules for filling to give the finished product;
wherein in step S3, a mass ratio of the thickener aqueous solution to the powder was 0.4:1.

### Example 4

A preparation method of a preservative-free cleansing and caring granule with low water activity was provided in Example 4, which differed from Example 1 only in that the components, except the thickener, and the amounts thereof were different. The cleansing and caring granule of this example comprised the following components in parts by weight: 5 parts of cetyl trimethyl ammonium chloride, 5 parts of cocamidopropyl betaine, 10 parts of sodium lauryl sulfate, 2 parts of sodium cocamoyl hydroxyethionate, 10 parts of modified corn starch, 1 part of magnesium stearate, 0.1 parts of sodium hyaluronate, 1 part of trehalose, 1 part of sorbitol, 0.0005 parts of Lactobacillus/rye flour ferment filtrate, 0.0005 parts of acanthus kernel oil, 0.0005 parts of Jojoba seed oil, 0.0005 parts of coconut oil, 0.1 parts of citric acid and 0.5 parts of daily essence. The thickener and the amount thereof, and the preparation method of the cleansing and caring granule were the same as those of Example 1.

### Example 5

A preparation method of a preservative-free cleansing and caring granule with low water activity was provided in Example 5, which differed from Example 1 only in that the components, except the thickener, and the amounts thereof were different. The cleansing and caring granule of this example comprised the following components in parts by weight: 20 parts of cetyl trimethyl ammonium chloride, 15 parts of cocamidopropyl betaine, 20 parts of sodium lauryl sulfate, 6 parts of sodium cocamoyl hydroxyethionate, 15 parts of modified corn starch, 5 parts of magnesium stearate, 1 part of sodium hyaluronate, 10 parts of trehalose, 10 parts of sorbitol, 0.0015 parts of Lactobacillus/rye flour ferment filtrate, 0.0015 parts of acanthus kernel oil, 0.0015 parts of jojoba seed oil, 0.0015 parts of coconut oil, 1 part of citric acid and 1.5 parts of daily essence. The thickener and the amount thereof, and the preparation method of the cleansing and caring granule were the same as those of Example 1.

### Comparative Example 1

A preparation method of a preservative-free cleansing and caring granule with low water activity was provided in this comparative example, which differed from Example 1 only in that a mass concentration of the thickener in the thickener aqueous solution was 0.1%, and the mass ratio of hydroxypropyl methyl cellulose/xanthan gum/chitosan in the thickener was 0.5:0.3:0.2. The remaining components and the amounts thereof, and the preparation method of the cleansing and caring granule were the same as those of Example 1.

### Comparative Example 2

A preparation method of a preservative-free cleansing and caring granule with low water activity was provided in this comparative example, which differed from Example 1 only in that a mass concentration of the thickener in the thickener aqueous solution was 1%, and the mass ratio of hydroxypropyl methyl cellulose/xanthan gum/chitosan in the thickener was 0.5:0.3:0.2. The remaining components and the amounts thereof and the preparation method of the cleansing and caring granule were the same as those of Example 1.

### Comparative Example 3

A preparation method of a preservative-free cleansing and caring granule with low water activity was provided in this comparative example, which differed from Example 1 only in that in step S3, the air temperature in the granulator was controlled at 70° C. All components and the amounts thereof, and the preparation method of the cleansing and caring granule were the same as those of Example 1.

### Comparative Example 4

A preparation method of a preservative-free cleansing and caring granule with low water activity was provided in this comparative example, which differed from Example 1 only in that in step S3, the air temperature in the granulator was controlled at 100° C. All components and the amounts thereof, and the preparation method of the cleansing and caring granule were the same as those of Example 1.

### Comparative Example 5

A preparation method of a preservative-free cleansing and caring granule with low water activity was provided in this comparative example, which differed from Example 1 only in that a mass ratio of the thickener aqueous solution to the powder was 0.1:1, and an operating frequency of the blower was controlled at 5 Hz. The remaining conditions were the same as those of Example 1.

### Comparative Example 6

A preparation method of a preservative-free cleansing and caring granule with low water activity was provided in this comparative example, which differed from Example 1 only in that a mass ratio of the thickener aqueous solution to the powder was 0.6:1, and an operating frequency of the blower was controlled at 60 Hz. The remaining conditions were the same as those of Example 1.

### Comparative Example 7

A preparation method of a preservative-free cleansing and caring granule with low water activity was provided in this comparative example, which differed from Example 1 only in that in step S3, an outlet air temperature of the granulator was controlled at 60° C., and a humidity was controlled at 50%. The remaining conditions were the same as those of Example 1.

### Comparative Example 8

A preparation method of a preservative-free cleansing and caring granule with low water activity was provided in this comparative example, which differed from Example 1 in that: the thickener was directly mixed with other components to give a mixture, the mixture was dried and ground into powder, then the powder was added to the granulator, the deionized water was evenly sprayed into the granulator by using a peristaltic pump, where a mass ratio of the deionized water to the powder was 0.3:1. All components and the amounts thereof, and the preparation method of the cleansing and caring granule were the same as those of Example 1.

### Comparative Example 9

A preparation method of a preservative-free cleansing and caring granule with low water activity was provided in this comparative example, which differed from Example 1 only in that a water-soluble polysaccharide, i.e., trehalose, instead of the thickener, was mixed with water to give a trehalose aqueous solution with a mass concentration of 0.5%, and then the trehalose aqueous solution was sprayed for granulation. The remaining components and the amounts thereof, and the preparation method of the cleansing and caring granule were the same as those of Example 1.

### Comparative Example 10

A preparation method of a preservative-free cleansing and caring granule with low water activity was provided in this comparative example, which differed from Example 1 only in that the components were different. In this comparative example, xanthan gum and chitosan were absent, and the thickener was hydroxypropyl methyl cellulose alone. The remaining components and the amounts thereof, and the preparation method of the cleansing and caring granule were the same as those of Example 1.

### Comparative Example 11

A preparation method of a preservative-free cleansing and caring granule with low water activity was provided in this comparative example, which differed from Example 1 only in that the components were different. In this comparative example, hydroxypropyl methyl cellulose and chitosan were absent, and the thickener was xanthan gum alone. The remaining components and the amounts thereof, and the preparation method of the cleansing and caring granule were the same as those of Example 1.

### Comparative Example 12

A preparation method of a preservative-free cleansing and caring granule with low water activity was provided in this comparative example, which differed from Example 1 only in that the components were different. In this comparative example, cetyltrimethyl ammonium chloride and sodium cocoyl hydroxyethylsulfonate were absent, the amount of cocamidopropyl betaine was increased to 20 parts by weight, and the amount of sodium lauryl sulfate was increased to 25 parts by weight, in order to keep the total amount of the surfactant unchanged. The remaining components and the amounts thereof, and the preparation method of the cleansing and caring granule were the same as those of Example 1.

### Comparative Example 13

A preparation method of a preservative-free cleansing and caring granule with low water activity was provided in this comparative example, which differed from Example 1 only in that the components were different. In this comparative example, cocamidopropyl betaine and sodium lauryl sulfate were absent, the amount of cetyltrimethyl ammonium chloride was increased to 30 parts by weight, and the amount of sodium cocoyl hydroxyethylsulfonate was increased to 15 parts by weight, in order to keep the amount of the surfactant unchanged. The remaining components and the amounts thereof, and the preparation method of the cleansing and caring granule were the same as those of Example 1.

### Test Example

### 1. Tests on Appearance State, Hardness and Dissolution Rate of Granules:

The cleansing and caring granules obtained in Examples 1-5 and Comparative Examples 1-13 were each taken as a sample, and subjected to tests on appearance state observation, hardness and dissolution rate. The test method of the dissolution rate was as follows: 2 g of each sample was weighed and placed in 100 mL of deionized water, the deionized water was stirred continuously by a stirrer with a stirring rate of 50 r/min, and the dissolution time of the sample in water was measured to evaluate the dissolution rate. The hardness of each of the cleansing and caring granules was tested by a hardness tester, and the hardness value (P value) was recorded in N. The determination method of the hardness could be carried out according to Chinese Pharmacopoeia 2005 edition. The results were shown in Table 1.

TABLE 1

| | Appearance | Hardness | Dissolution |
|---|---|---|---|
| Sample | state | value (N) | time/s |
| Example 1 | Uniform in size, | 327 | 35 |
| | structurally | | |
| | intact, and dry | | |
| | without | | |
| | agglomeration | | |
| Example 2 | Uniform in size, | 305 | 54 |
| | structurally | | |
| | intact, and dry | | |
| | without | | |
| | agglomeration | | |
| Example 3 | Uniform in size, | 318 | 46 |
| | structurally | | |
| | intact, and dry | | |
| | without | | |
| | agglomeration | | |
| Example 4 | Uniform in size, | 322 | 40 |
| | structurally | | |
| | intact, and dry | | |
| | without | | |
| | agglomeration | | |
| Example 5 | Uniform in size, | 313 | 45 |
| | structurally | | |
| | intact, and dry | | |
| | without | | |
| | agglomeration | | |
| Comparative | Non-uniform in | 231 | 63 |
| Example 1 | size | | |
| Comparative | Non-uniform in | 284 | 69 |
| Example 2 | size and | | |
| | agglomerated | | |
| Comparative | Non-uniform in | 228 | 62 |
| Example 3 | size, wet and | | |
| | agglomerated | | |
| Comparative | Uniform in size | 297 | 86 |
| Example 4 | | | |
| Comparative | Non-uniform in | 239 | 65 |
| Example 5 | size and | | |
| | structurally | | |
| | defective | | |
| Comparative | Non-uniform in | 286 | 68 |
| Example 6 | size and | | |
| | agglomerated | | |
| Comparative | Non-uniform in | 261 | 73 |
| Example 7 | size and | | |
| | agglomerated | | |
| Comparative | Non-uniform in | 241 | 68 |
| Example 8 | size | | |
| Comparative | Non-uniform in | 254 | 66 |
| Example 9 | size and | | |
| | agglomerated | | |
| Comparative | Uniform in size | 273 | 71 |
| Example 10 | and dry without | | |
| | agglomeration | | |
| Comparative | Uniform in size | 261 | 73 |
| Example 11 | and dry without | | |
| | agglomeration | | |
| Comparative | Uniform in size | 293 | 65 |
| Example 12 | and structurally | | |
| | intact relatively | | |
| Comparative | Uniform in size | 296 | 67 |
| Example 13 | and structurally | | |
| | intact relatively | | |

As can be seen from the results in Table 1, the cleansing and caring granules prepared in the disclosure are uniform in size and possess relatively high hardness, which can effectively avoid the risk of breakage and fragmentation of the granules during transportation or storage. Besides, the cleansing and caring granules possess better solubility and can be quickly dissolved during use. In particular, the cleansing and caring granule prepared in Example 1 possesses the highest hardness and solubility. However, in Comparative Examples 1-9, since the parameters of the preparation methods exceed the limited ranges of the disclosure, the prepared cleansing and caring granules are non-uniform in size and agglomerate among granules, which is not conducive to the practical production. Compared with the cleansing and caring granules prepared in Examples 1-5, the granules prepared in Comparative Examples 1-9 exhibit significantly reduced hardness, lower compactness, and poor solubility. In Comparative Examples 10 and 11, due to the absence of any two of hydroxypropyl methyl cellulose, xanthan gum and chitosan, the granule compactness cannot be effectively improved, the granule hardness decreases to a certain extent, and the solubility of the granules has significantly deteriorated.

The sole FIGURE showed the appearance images of the cleansing and caring granules prepared in Example 1 (A) and in Comparative Example 1 (B) respectively. As can be seen from the sole FIGURE, the cleansing and caring granule prepared in Example 1 of the disclosure is uniform in size, structurally intact and high in hardness, while the cleansing and caring granule prepared in Comparative Example 1 is non-uniform in size distribution, non-uniform in size, low in compactness and fragile, which is not conductive to the storage and transportation of the cleansing and caring granules.

### 2. Water Activity (Aw) Test:

The water activity of the cleansing and caring granules obtained in Example 1, Comparative Examples 1-13 were tested. The test method was carried out using a water activity meter, according to United States Pharmacopeia—National Formulary (USP-NF) General Chapter 922: Water Activity. The results were shown in Table 2.

TABLE 2

| Sample | Water activity |
|---|---|
| Example 1 | 0.29 |
| Comparative | 0.76 |
| Example 1 | |
| Comparative | 0.65 |
| Example 2 | |
| Comparative | 0.85 |
| Example 3 | |
| Comparative | 0.73 |
| Example 4 | |
| Comparative | 0.74 |
| Example 5 | |
| Comparative | 0.68 |
| Example 6 | |
| Comparative | 0.76 |
| Example 7 | |
| Comparative | 0.82 |
| Example 8 | |
| Comparative | 0.71 |
| Example 9 | |
| Comparative | 0.56 |
| Example 10 | |
| Comparative | 0.58 |
| Example 11 | |
| Comparative | 0.59 |
| Example 12 | |
| Comparative | 0.61 |
| Example 13 | |

Water activity has an important impact on the property, stability and usage effect of the product. In general, low water activity is conductive to improving the stability of the solid cleansing and caring granule, which can avoid degradation or inefficiency of the active ingredients therein. Moreover, low water activity is conductive to controlling the generation of foam, achieving more stable foam quantity and foam quality. This enables solid cleansing and caring granule to produce more uniform foam during use, and the foam is easy to rinse without residue.

As can be seen from the results in Table 2, the cleansing and caring granules prepared in the disclosure can achieve relatively low water activity, especially the water activity of the cleansing and caring granule prepared in Example 1 reaches 0.29, which can fully ensure the stability and overall performance of the cleansing and caring granule, so that the cleansing and caring granule has an anti-corrosion effect without adding preservatives. However, in Comparative Examples 1-7, since the parameters of the preparation methods of the cleansing and caring granules exceed the optimized ranges of this disclosure, the water activity of the granules increases more significantly. In Comparative Example 8, the thickener is directly mixed with other components, and in Comparative Example 9, trehalose with similar adhesiveness is used instead of the thickener described in the disclosure for granulation, the water activity of the finally prepared cleansing and caring granules is significantly increased. In Comparative Examples 12-13, since the specific surfactant components are absent or replaced with other surfactant components, the water activity of the prepared cleansing and caring granules increases to a certain extent compared to Example 1.

### 3. Sensory Evaluation:

The cleansing and caring granules obtained in Examples 1-5 and Comparative Examples 12-13 were subjected to sensory evaluation.

70 subjects aged 20-60 years with normal skin conditions were randomly selected, including 10 males and 60 females. The subjects were randomly divided into seven groups with 10 subjects in each group. The solid cleansing and caring granules prepared in Examples 1-5 and Comparative Examples 12-13 were each tested respectively for shampoo performance evaluation. The samples were each used for 2 weeks. Based on their experience, the subjects evaluated the samples based on a scoring system. Evaluation items included hair cleansing and decontamination efficacy, foam richness, easy rinsing without residue effects. A 10-point scale was adopted for scoring, 1 score means the worst and 10 scores mean the best. The evaluation results were represented by the average scores of all subjects in each group, as shown in Table 3. The higher the average score, the better the performance.

TABLE 3

| | Cleansing and | Foam | Easy rinsing |
|---|---|---|---|
| Sample | decontamination | richness | without residue |
| Example 1 | 9.5 | 9.7 | 9.6 |
| Example 2 | 8.8 | 9.0 | 9.3 |
| Example 3 | 9.5 | 9.6 | 8.9 |
| Example 4 | 9.3 | 9.6 | 9.5 |
| Example 5 | 9.3 | 9.4 | 9.4 |
| Comparative | 7.2 | 6.3 | 7.5 |
| Example 12 | | | |
| Comparative | 6.1 | 6.5 | 7.3 |
| Example 13 | | | |

As can be seen from the results in Table 3, the cleansing and caring granules prepared in the disclosure achieve a balance in aspects of cleansing efficacy, foam stability and easy rinsing in actual use process, and can provide consumers with more ideal usage experience.

Finally, it should be noted that the above embodiments are intended to illustrate the technical solutions of the disclosure and not to limit the protection scope thereof. Although the disclosure is described in detail by reference to preferred embodiments, persons of ordinary skill in the art should understand that the technical solution of the disclosure can be modified or equivalently replaced without deviating from the substance and scope of the technical solution of the disclosure.

What is claimed is:

1. A preparation method of a preservative-free cleansing and caring granule with low water activity, comprising the following steps:
S1. weighing the following components in parts by weight: 22-61 parts of a surfactant, 10-15 parts of a filler, 1-5 parts of an anti-caking agent, 2.1-21 parts of a moisturizer, 0.1-1 part of a pH regulator and 0.5-1.5 parts of a fragrance, mixing the weighed components evenly to give a mixture, subjecting the mixture to drying and grinding to give powder;
S2. weighing a thickener, and mixing the thickener with water to give a thickener aqueous solution;
S3. adding the powder obtained in step S1 to a granulator, then spraying the thickener aqueous solution obtained in step S2 into the granulator to prepare a granule as the cleansing and caring granule, during which an air temperature in the granulator is controlled at 80° C.-90° C.;
wherein a mass concentration of the thickener aqueous solution is 0.3%-0.7%.

2. The preparation method according to claim 1, wherein in step S3, a mass ratio of the thickener aqueous solution to the powder is (0.2-0.4):1.

3. The preparation method according to claim 2, wherein the mass ratio of the thickener aqueous solution to the powder is 0.3:1.

4. The preparation method according to claim 1, wherein in step S3, the air temperature in the granulator is controlled by a blower, and an operating frequency of the blower is controlled at 15-50 Hz; and/or,
an outlet air temperature of the granulator is controlled at 50° C. or lower, and a humidity is controlled at 20-40%.

5. The preparation method according to claim 1, wherein the mass concentration of the thickener in the thickener aqueous solution is 0.5%.

6. The preparation method according to claim 1, wherein the preservative-free cleansing and caring granule comprises the following components in parts by weight: 45 parts of the surfactant, 12 parts of the filler, 3 parts of the anti-caking agent, 15 parts of the moisturizer, 0.5 parts of the pH regulator and 1 part of the fragrance.

7. The preparation method according to claim 1, wherein the thickener comprises hydroxypropyl methyl cellulose, xanthan gum and chitosan, and a mass ratio of the hydroxypropyl methyl cellulose/xanthan gum/chitosan is (0.1-1):(0.1-0.5):(0.1-0.3).

8. The preparation method according to claim 7, wherein the mass ratio of the hydroxypropyl methyl cellulose/xanthan gum/chitosan is 0.5:0.3:0.2.

9. The preparation method according to claim 1, wherein the surfactant comprises cetyltrimethyl ammonium chloride, cocamidopropyl betaine, sodium lauryl sulfate and sodium cocamyl hydroxyethylsulfonate, and a mass ratio of cetyltrimethyl ammonium chloride/cocamidopropyl betaine/sodium lauryl sulfate/sodium cocamyl hydroxyethylsulfonate is (5-20):(5-15):(10-20):(2-6).

10. The preparation method according to claim 1, wherein the filler comprises at least one of a modified corn starch, microcrystalline cellulose and polyvinyl alcohol; and/or,
the anti-caking agent comprises magnesium stearate or silicon dioxide; and/or,
the moisturizer comprises at least one of sodium hyaluronate, trehalose and sorbitol; and/or,
the pH regulator comprises citric acid; and/or,
the fragrance comprises an essence.

11. The preparation method according to claim 6, wherein the surfactant comprises cetyltrimethyl ammonium chloride, cocamidopropyl betaine, sodium lauryl sulfate and sodium cocamyl hydroxyethylsulfonate, and a mass ratio of cetyltrimethyl ammonium chloride/cocamidopropyl betaine/sodium lauryl sulfate/sodium cocamyl hydroxyethylsulfonate is (5-20):(5-15):(10-20):(2-6).

12. The preparation method according to claim 6, wherein the filler comprises at least one of a modified corn starch, microcrystalline cellulose and polyvinyl alcohol; and/or,
the anti-caking agent comprises magnesium stearate or silicon dioxide; and/or,
the moisturizer comprises at least one of sodium hyaluronate, trehalose and sorbitol; and/or,
the pH regulator comprises citric acid; and/or,
the fragrance comprises an essence.
